(19) United States
(12) Patent Application Publication
(10) Pub. No.: US 2019/0035503 A1
(43) Pub. Date: Jan. 31, 2019

(54) Method And System For Task Management And Communication

(71) Applicant: TaskUnite Inc., Santa Monica, CA (US)

(72) Inventors: Bianca Gonzalez, Santa Monica, CA (US); Mythri Papolu, Pleasanton, CA (US)

(21) Appl. No.: 16/050,927

(22) Filed: Jul. 31, 2018

Related U.S. Application Data
(60) Provisional application No. 62/539,227, filed on Jul. 31, 2017.

(51) Int. Cl. G16H 40/20; G16H 10/60; H04W 4/021

(52) U.S. Cl. CPC G16H 40/20; H04W 4/021; G16H 10/60

(57) ABSTRACT

Systems and techniques for task management and communication are disclosed. In one implementation, input indicating configurations to use to generate a task is received from a first client device. The task is generated using the configurations, in which the task is indicative of one or more actions to perform with respect to a patient of a health care system. A task record associated with the task is stored within a database of the health care system. The task record indicates a user of the health care system to which the task is assigned. Thereafter, further input indicating a change in status of the task is received from a second client device. The task record is updated according to the second input. A notification indicative of the updated task record is then transmitted to the first client device. Other implementations include using a smart recommendation engine, processing supplies, and more.

### CROSS-REFERENCE TO RELATED APPLICATION(S)

This disclosure claims priority to and the benefit of U.S. Provisional Application Patent Ser. No. 62/539,227, filed Jul. 31, 2017, the entire disclosure of which is hereby incorporated by reference.

### TECHNICAL FIELD

This disclosure relates to methods and systems for task management and communication.

### BACKGROUND

Workflow and task inefficiencies in the health system industry are often attributed to excess labor, poor charge capture, and leakage of costly medical supplies. Currently, healthcare administrative costs account for $200 billion of healthcare spending in the United States alone. Revenue leakage and poor charge capture from inefficient healthcare workflows accounts for $5 billion annually. Additionally, the United States spend on administrative costs is generally more than double international averages. According to the Organisation for Economic Co-operation and Development, poor coordination between health care providers and duplication of tasks contribute to inflated administrative costs. Therefore, there is a need for health care task management and communication that is cost-effective and efficient.

### SUMMARY

Disclosed herein are, inter alia, systems and techniques for task management and communication. In one implementation, an apparatus for health care task management and communication is provided. The apparatus comprises a server device including a processor, a memory, a storage, and a network interface. The storage includes a database that stores data associated with application software for a health care system. The processor executes instructions stored in the memory to perform operations of the application software. The operations include receiving, using the network interface, first input from a first client device. The first input indicates configurations to use to generate a task. The operations further include generating the task using the configurations. The task is indicative of one or more actions to perform with respect to a patient of the health care system. The operations further include, responsive to generating the task, storing a task record associated with the task within the database. The task record indicates a user of the health care system to which the task is assigned. The operations further include, subsequent to storing the task record within the database, receiving second input from a second client device. The second input indicates a change in status of the task. The operations further include updating the task record according to the second input. The operations further include transmitting, using the network interface, a notification indicative of the updated task record to the first client device.

In another implementation, a method for health care task management and communication is provided. The method includes generating virtual groups using health care system data stored within a database of a health care system. Each of the virtual groups includes at least one patient. A record of each patient is stored within the database. The method further includes creating a virtual team for each of the virtual groups. Each virtual team includes at least one user of the health care system. The method further includes, for at least one patient within each of the virtual groups, generating at least one task using associated patient data stored within the database. The method further includes assigning the at least one task for the patient to a user of the virtual team corresponding to the virtual group that includes the patient. The assigning uses a matching mechanism that processes, for the users within the virtual team corresponding to the virtual group that includes the patient, one or both of geofencing location coordinates of the users or availability status of the users. The method further includes, subsequent to the assigning, receiving data indicating a completion of the at least one task by the user to which the at least one task is assigned. The method further includes storing the data indicating the completion of the at least one task by the at least one user within the database.

In yet another implementation, a non-transitory computer readable medium comprising instructions that, when executed by a processor, cause the processor to perform operations for health care task management and communication is provided. The operations include defining a virtual group including patients of a health care system. The operations further include generating a task associated with at least one patient of the virtual group. The operations further include assigning the task to a user of the health care system using a matching mechanism. The operations further include receiving information indicating a completion of the task by the user.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a block diagram of an example of a system for task management and communication.

FIG. 2 is a block diagram of an example of an internal configuration of a computing device of a system for task management and communication.

FIG. 3 is an illustration of an example of authentication and authorization functionality of a software application for task management and communication.

FIGS. 4A-B are illustrations of an example of a form for creating and displaying team data within a software application for task management and communication.

FIGS. 5A-B are illustrations of an example of a workflow for task creation using a software application for task management and communication.

FIG. 6 is an illustration of an example of a form for requesting supplies within a software application for task management and communication.

FIGS. 7A-B are illustrations of an example of a workflow for task response using a software application for task management and communication.

FIG. 8 is an illustration of an example of a form for displaying task records within a software application for task management and communication.

FIG. 9 is an illustration of an example of data and analytical functionality of a software application for task management and communication.

FIG. 10 is a diagram shown example functionality of a smart recommendation engine of a software application for task management and communication.

FIG. 11 is a flowchart of an example of a technique for task management and communication.

FIG. 12 is a flowchart of an example of a technique for task management and communication.

### DETAILED DESCRIPTION

Health care task management and communication generally refers to the management and communication of tasks to be performed by a healthcare team. Conventional healthcare team communication workflows and tasks use to paper checklists or limited software functionality directly associated with electronic medical records. As a result, tasks may be performed incorrectly (e.g., for the wrong patient, using the wrong supplies, etc.), be assigned to an unskilled team member, or remain pending even after a stated deadline for completion.

In some cases, a healthcare team may use tools (e.g., conventional workflow management systems) to assist with the task management and communication between team members; however, such tools fail for a number of reasons. For example, these conventional tools do not dispatch real-time or near real-time alerts associated with specific patients, such as to client devices used by the team members (e.g., mobile devices such as smart phones, tablets, and smart watches). Moreover, these conventional tools do account for supply management and do not have the functionality to account for supplies used for specific patient-related tasks in real-time or near real-time. Rather, typical health care organizations lack data and predictive analytics tools to guide actionable decisions in optimizing staffing levels and managing the supply chain.

The implementations of this disclosure address problems such as these using a software application for task management and communication. This software application, which may be or otherwise refer to the TaskUnite™ application system, addresses problems such as the foregoing by providing a platform used for creating a task, delegating a task to clinical team members, associating a task with patients or patient pools with administrator-approved access, creating clinical teams with administrator-approved access to patient pools (creating a list of authorized users), deleting a task, setting an alert or reminder for a task, changing a status of the task, and/or associating the task with supplies or equipment used to complete the task.

The implementations of this disclosure further describe that supply and equipment association with a task will be implemented through bar code or other code reading capabilities on client devices used to access the software application. Such functionality may also be usable for automatically detecting inventory levels of supplies and automating the re-ordering of inventory from suppliers, such as without manual user intervention. Data is secured to private cloud or local servers with automatic backup sync capabilities that do not need internet connectivity at all times.

The implementations of this disclosure describe systems and techniques which can be used to communicate task status updates and supply usage among one or multiple persons in a health care team that may be located in, but are not limited to, ambulatory clinics, inpatient hospital settings, home care settings, public health facilities, retail clinics, skilled nursing facilities, insurance companies, government health facilities, medical supply companies, and medical device companies. However, the systems and techniques disclosed herein may also be used outside of the healthcare (e.g., medical) context. For example, the systems and techniques of this disclosure may be implemented in other industries in which tasks may be assigned and performed in teams.

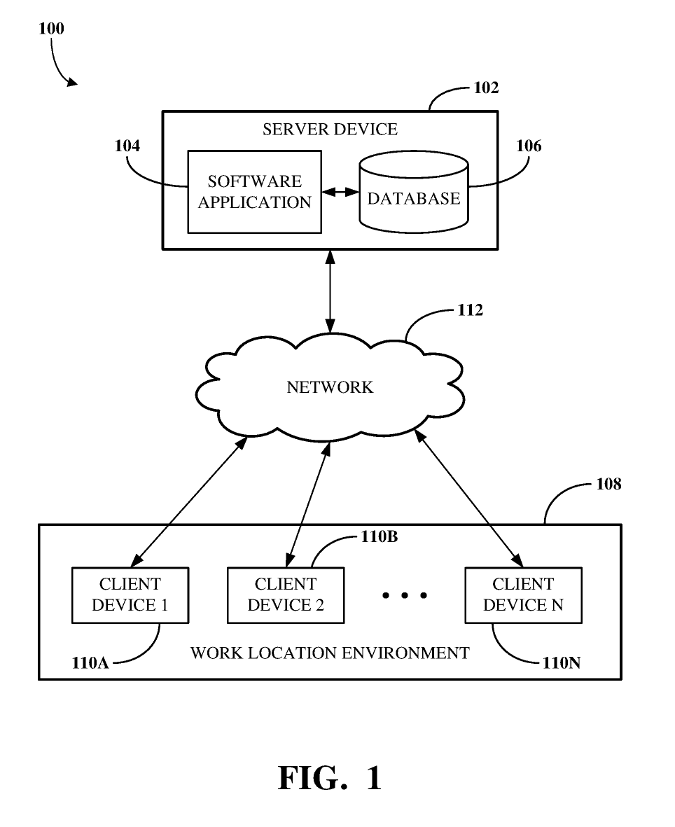

FIG. 1 is a block diagram of an example of a system for task management and communication 100, referred to hereafter as the system 100. The system 100 includes a server device 102. The server device 102 is a computing device that hosts or otherwise makes available a software application 104 and a database 106, in which the database 106 stores data used by the software application 104. The server device 102 may include an application server and/or a database server. The server device 102 may be a server located in a rack, such as of a data center, or a standalone server device.

The software application 104 may be a software application of a health care system. The software application 104 includes instructions or other code which, when executed, interpret, run, or otherwise processed at or using the server device 102, implement functionality for the systems and techniques described herein. In particular, the software application 104 is a software application for task management and communication. The software application 104 may be used to track, create, and manage tasks in real-time or near real-time through secure messaging or using other notifications.

The software application 104 may, for example, be a digital health software application used to share, create, delegate, and/or follow-up on tasks among users (e.g., members of a health care team, for example, nursing staff at a hospital). The software application 104 may further include functionality for supply and equipment association with a task, such as through bar code or other code reading functionality. Such supply and equipment association functionality may be used for automatically detecting inventory levels of supplies and automating the re-ordering of inventory from suppliers.

The server device 102 includes a processor configured to execute instructions stored in a memory of the server device 102 to perform some or all of the functionality of the software application 104. Performing functionality of the software application 104 may include using data stored in the database 106. The database 106 may be a relational database, an object database, an XML database, or a database of another suitable database type. The data stored in the database 106 may include records associated with tasks created, updated, or otherwise managed and communicated using the software application 104.

The software application 104 is accessed by devices within a work location environment 108. The work location environment 108 may, for example, refer to a physical location or set of physical locations common to users of the devices that access the software application 104. For example, the work location environment 108 may refer to a hospital or another medical care or treatment facility. The work location environment 108 may represent a group of users who are able to assign, delegate, and perform tasks in connection with one another. Separate instances of the software application 104 may be available to different work location environments, such as to prevent the data and configurations of the work location environment 108 from being made available to another work location environment.

The work location environment 108 includes client devices 110A, 110B, and 110N. The one or more of the client devices 110A, 110B, and 110N may, for example, be a mobile device, such as a smart phone, tablet, laptop, or the like. The one or more of the client devices 110A, 110B, and 110N runs software (e.g., client-side software) used to communicate with the server device 102. For example, the software application 104 may be a software application accessed using a client-side application that connects the one or more of the client devices 110A, 110B, and 110N to the server device 102. In another example, the software application 104 may be a web-based software application accessed using a web browser running at one of the client devices 110A, 110B, and 110N.

The client devices 110A, 110B, and 110N communicates with the server device 102 over a network 112. The network 112 may, for example, be a local area network, a wide area network, a machine-to-machine network, a virtual private network, or another public or private network. For example, the software application 104 may be configured, deployed, or otherwise run on the server device 102 based on instructions, commands, or other data received one or more of the client devices 110A, 110B, and 110N over the network 112.

The operation of the software application 104 includes the generation or rendering and subsequent display of one or more graphical user interfaces (GUIs). As used herein, the displaying of a GUI of the software application 104 refers to the outputting or other rendering of the GUI on one of the client devices 110A, 110B, or 110N, or on another client device or computing device used to run, execute, interpret, or otherwise access the software application 104 or a version thereof.

As used herein, a GUI of a software application for task management and communication (e.g., the software application 104) can comprise part of a software GUI constituting data that reflect information ultimately destined for display on a computing device, such as one of the client devices 110A, 110B, or 110N. For example, the data can contain rendering instructions for bounded graphical display regions, such as windows, or pixel information representative of controls, such as buttons and drop-down menus. The rendering instructions can, for example, be in the form of HTML, SGML, JavaScript, Jelly, AngularJS, or other text or binary instructions for generating a GUI on a display that can be used to generate pixel information. A structured data output of one computing device can be provided to an input of the display so that the elements provided on the display screen represent the underlying structure of the output data.

Implementations of the system 100 may differ from what is shown and described with respect to FIG. 1. In some implementations, the server device 102 may be a local server device connected using an Intranet or other local connection between the client devices 110A, 110B, and 110N, such as without requiring Internet access. For example, the local server device may be a hub (e.g., a hardware hub or a software hub) that processes and routes requests and commands between the instances of the software application running at some or all of the client devices 110A, 110B, and 110N.

In some implementations, the software application 104 may be locally installed on some or all of the client devices 110A, 110B, and 110N. For example, the server device 102 may run a server-side application (e.g., a full software application or a set of scripts used to run the software application). The client devices 110A, 100B, and 110N store the instructions for running the software application locally, such as on a non-volatile memory, storage device, or other non-transitory computer storage medium.

In some implementations, the software application 104 may represent software implemented using a cloud computing environment. For example, the software application may represent a Software-as-a-Service (SaaS) application or like functionality hosted within a cloud computing environment for access by computing devices of one or more tenants (e.g., the work location environment 108). In such an implementation, the server device may include one or more application nodes and/or database nodes for implementing the instance or instances of the SaaS application.

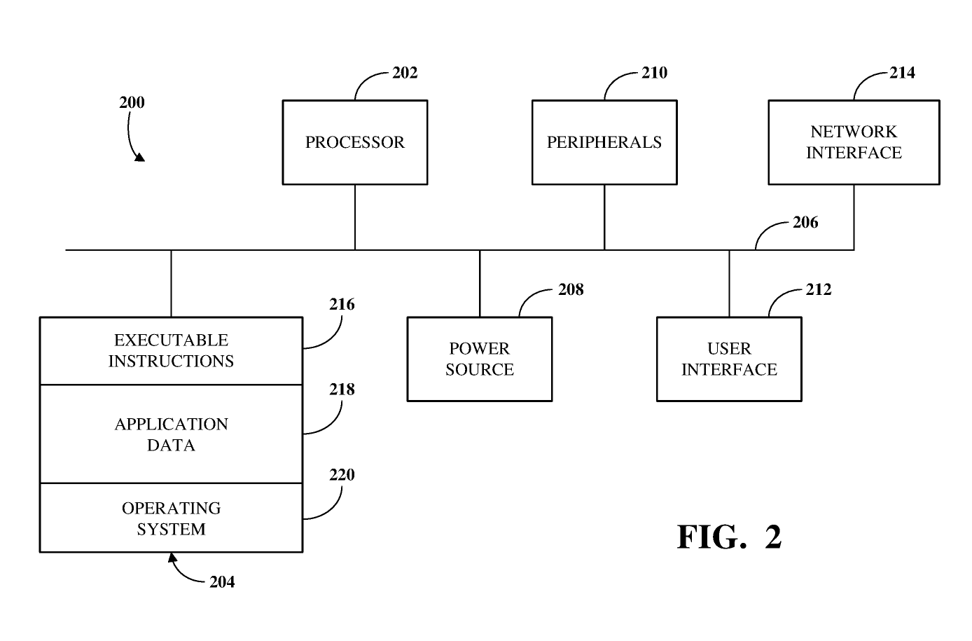

FIG. 2 is a block diagram of an example of an internal configuration of a computing device 200 of a system for task management and communication, such as the system 100 shown in FIG. 1. For example, the computing device 200 may be the server device 102 or one of the client devices 110A, 110B, or 110N shown in FIG. 1. The computing device 200 includes a processor 202, a memory 204, a bus 206, a power source 208, peripherals 210, a user interface 212, and a network interface 214. One of more of the memory 204, the power source 208, the peripherals 210, the user interface 212, or the network interface 214 can communicate with the processor 202 via the bus 206.

The processor 202 is a central processing unit, such as a microprocessor, and can include single or multiple processors having single or multiple processing cores. Alternatively, the processor 202 can include another type of device, or multiple devices, now existing or hereafter developed, configured for manipulating or processing information. For example, the processor 202 can include multiple processors interconnected in any manner, including hardwired or networked, including wirelessly networked. For example, the operations of the processor 202 can be distributed across multiple devices or units that can be coupled directly or across a local area or other suitable type of network. The processor 202 can include a cache, or cache memory, for local storage of operating data or instructions.

The memory 204 includes one or more memory components, which may each be volatile memory or non-volatile memory. For example, the volatile memory of the memory 204 can be random access memory (RAM) (e.g., a DRAM module, such as DDR SDRAM) or another form of volatile memory. In another example, the non-volatile memory of the memory 204 can be a disk drive, a solid state drive, flash memory, phase-change memory, or another form of non-volatile memory configured for persistent electronic information storage. The memory 204 may also include other types of devices, now existing or hereafter developed, configured for storing data or instructions for processing by the processor 202.

The memory 204 can include data for immediate access by the processor 202. For example, the memory 204 can include executable instructions 216, application data 218, and an operating system 220. The executable instructions 216 can include one or more application programs, which can be loaded or copied, in whole or in part, from non-volatile memory to volatile memory to be executed by the processor 202. For example, the executable instructions 216 can include instructions for performing some or all of the techniques of this disclosure.

The application data 218 can include user data, database data (e.g., database catalogs or dictionaries), or the like. The operating system 220 can be, for example, Microsoft Windows®, Mac OS X®, or Linux®; an operating system for a small device, such as a smartphone or tablet device; or an operating system for a large device, such as a mainframe computer.

The power source 208 includes a source for providing power to the computing device 200. For example, the power source 208 can be an interface to an external power distribution system. In another example, the power source 208 can be a battery, such as where the computing device 200 is a mobile device or is otherwise configured to operate independently of an external power distribution system.

The peripherals 210 includes one or more sensors, detectors, or other devices configured for monitoring the computing device 200 or the environment around the computing device 200. For example, the peripherals 210 can include a geolocation component, such as a global positioning system location unit. In another example, the peripherals can include a temperature sensor for measuring temperatures of components of the computing device 200, such as the processor 202.

The user interface 212 includes one or more input interfaces and/or output interfaces. An input interface may, for example, be a positional input device, such as a mouse, touchpad, touchscreen, or the like; a keyboard; or another suitable human or machine interface device. An output interface may, for example, be a display, such as a liquid crystal display, a cathode-ray tube, a light emitting diode display, or other suitable display.

The network interface 214 provides a connection or link to a network, for example, a local area network, a wide area network, a machine-to-machine network, a virtual private network, or another public or private network. The network interface 214 can be a wired network interface or a wireless network interface. The computing device 200 can communicate with other devices via the network interface 214 using one or more network protocols, such as using Ethernet, TCP, IP, power line communication, Wi-Fi, Bluetooth®, infrared, GPRS, GSM, CDMA, Z-Wave, ZigBee, another protocol, or a combination thereof.

Implementations of the computing device 200 may differ from what is shown and described with respect to FIG. 2. In some implementations, the computing device 200 can omit the peripherals 210. In some implementations, the memory 204 can be distributed across multiple devices. For example, the memory 204 can include network-based memory or memory in multiple clients or servers performing the operations of those multiple devices. In some implementations, the application data 218 can include functional programs, such as a web browser, a web server, a database server, another program, or a combination thereof.

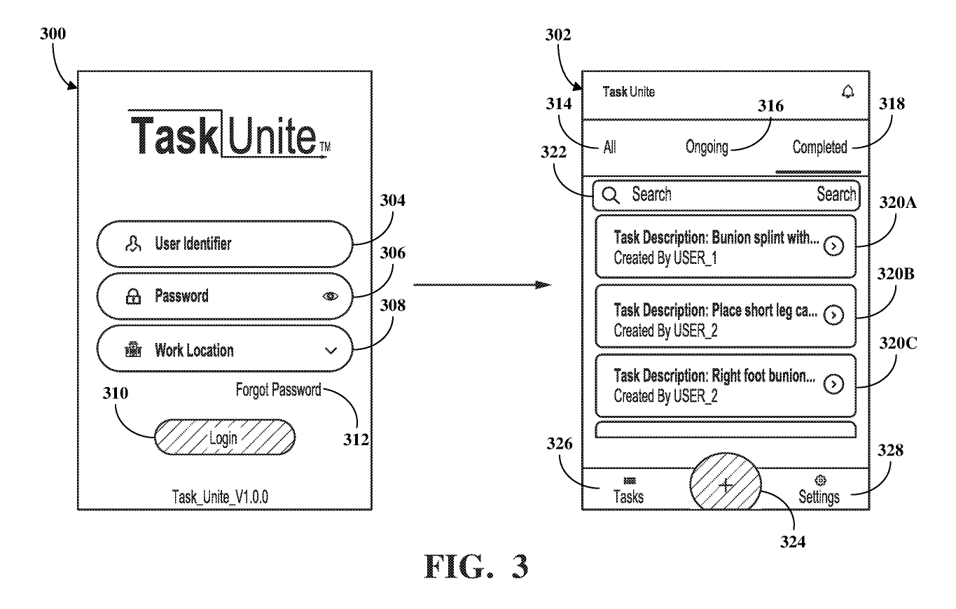

FIG. 3 is an illustration of an example of authentication and authorization functionality of a software application for task management and communication, which may, for example, be the software application 104 shown in FIG. 1. In particular, FIG. 3 shows GUIs of the software application including a first GUI 300 for receiving user input for authentication and authorization and a second GUI 302 for displaying records subsequent to a successful authentication and authorization.

The first GUI 300 includes fields 304, 306, and 308 for receiving user input. In particular, and respectively, the fields 304, 306, and 308 receive user input for a user identifier (e.g., an email address, user name, or other unique identifier for the user to be authenticated), a password for authorizing the user to access the requested user account (e.g., an alpha-numeric character string), and a work location (e.g., a hospital or other physical location at which services organized using the software application may be provided).

After entering input within each of the fields 304, 306, and 308, the user may interact with a login element 310 to process the input for authentication and authorization. The first GUI 300 may further include a “forgot password” element 312 for allowing the user to verify his or her identity, such as to reset the login password for his or her user account.

For example, the input may be locally authenticated and authorized, such as in implementations where the software application is locally executed (e.g., without a network connection or otherwise without communication with another computing device, for example, the server device 102 shown in FIG. 1). In another example, the input may be transmitted to another computing device, such as the server device 102, for authentication and authorization.

Interacting with the login element 310 may cause a secure sockets layer (SSL) request or other encrypted request (e.g., using transport layer security (TLS)) to be transmitted over a network, such as the network 112 shown in FIG. 1. The input entered into the fields 304, 306, and/or 308 may be used for two-factor authorization (2FA). For example, the input entered into the fields 304, 306, and/or 308 may be stored in a database of an authentication service that uses or otherwise supports 2FA.

The user account to which access is authenticated and authorized is associated with task records representing ongoing tasks, completed tasks, or both. The second GUI 302 represents an example of a GUI the user may see within the software application upon being granted access to his or her user account. The second GUI 302 includes selection elements 314, 316, and 318 for navigating between categories of task records. For example, and respectively, the selection elements 314, 316, and 318 may be selected to view task records associated with all tasks, task records associated with ongoing tasks, or task records associated with completed tasks. Selecting one of the selection elements 314, 316, or 318 results in the corresponding task records being displayed on the second GUI 302.

Examples of task records displayed within the second GUI 302 are shown at 320A, 320B, and 320C. For example, the display of a task record may be represented by a user interface element describing the task, identifying the user who created the task, indicating the creation date and/or intended completion date for the task, or other information associated with the task. A search bar 322 allows the user to search for a task record, such as based on keyword or other information related to a task record.

Upon user account authentication and authorization, the software application (e.g., using the second GUI 302 or otherwise) may identify a title of the user. Depending on the title of the user, the second GUI 302 may include additional user interface elements. For example, the second GUI 302 may include one or more of a new task element 324 for allowing the user to create a new task, a task delegation element 326 for allowing the user to assign one or more tasks to another user of the software application, or a settings element 328 for configuring aspects of the software application.

For example, the title of the user may be one of an administrator, a manager, a service provider (e.g., in a medical context, a nurse, doctor, physical therapist, pharmacist, etc.), a staff member, or another title. The additional user interface for creating new tasks, delegating tasks, configuring settings, or otherwise may be restricted to users having a particular title. The permissions and types of user interface elements available to a given user within the software application may be defined according to the settings of the team to which the user is assigned or the location at which the user works.

Other examples of additional user interface elements based on the user title may be available. For example, if the user is identified as an administrator, the second GUI 302 may include user interface elements to assign staff roles, manage tasks, view and conduct analytics, create tasks, delegate tasks, create teams, configure settings, etc. For example, the administrator may create teams by prompting email invites to other authorized users. The administrator may also have security privileges to invite and approve users to be included on teams and access specific client information (e.g., in a medical context, patient information or patient pools).

Implementations of the authentication and authorization functionality of a software application for task management and communication may differ from what is shown and described with respect to FIG. 3. In some implementations, the first GUI 300 may receive input other than by using the fields 304, 306, and 308. For example, a fingerprint or other biometric input may be received and verified against stored user account data to authenticate and authorize a user account access. In another example, a security code other than as used in 2FA may be used to authenticate and authorize the user account access, such as instead of or in addition to the input received using the fields 304, 306, and 308.

In some implementations, the software application (e.g., by using the first GUI 300, the second GUI 302, or another GUI or software aspect) may request or require that the user identify his or her title as part of the authentication and authorization process. For example, the software application may prompt the user to identify himself or herself as one of an administrator, a manager, a service provider, a staff member, or another title. In some implementations, the title may be expressed in multiple layers. For example, a first layer may reflect that the user is a service provider and the second layer may reflect the type of service provider the user is (e.g., a nurse or a doctor).

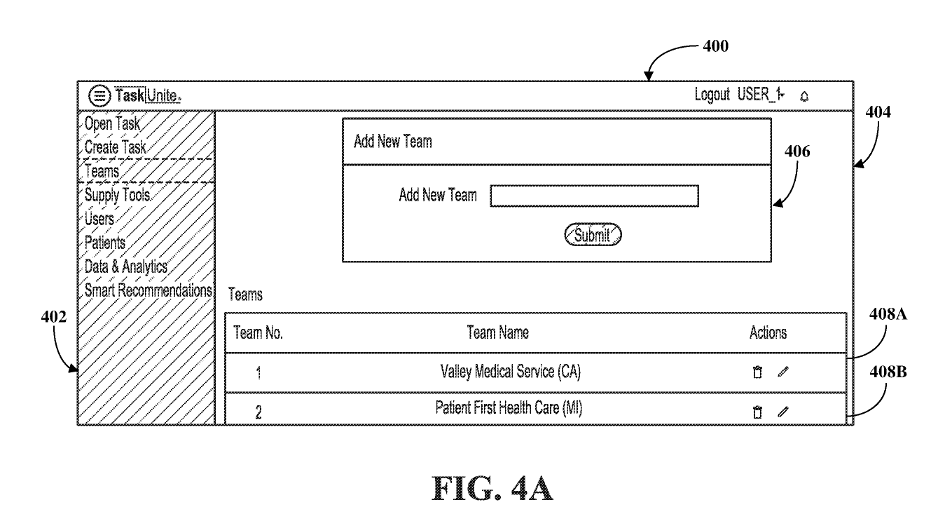

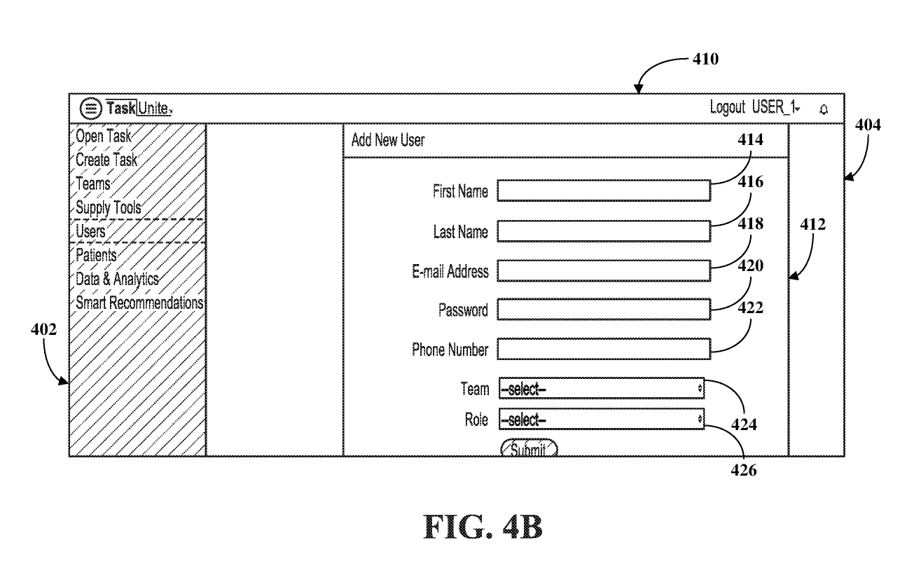

FIGS. 4A-B are illustrations of an example of a form for creating and displaying team data within a software application for task management and communication, which may, for example, be the software application 104 shown in FIG. 1. Referring first to FIG. 4A, a form 400 is shown. The form 400 includes a frame 402 and a body 404. The frame 402 includes user interface elements selectable to navigate between pages of the software application. For example, the pages can refer to forms or other functionality or aspects of the software application relating to opening tasks, creating tasks, teams, supply tools, users, patients, data and analytics, and smart recommendations. The body 404 includes a sub-form 406. The sub-form 406 includes a field for receiving input used to create a new team. The body 404 further includes a visual representation of records associated with teams which have been created. For example, a first record 408A indicates information associated with a first team and a second record 408B indicates information associated with a second team.

Referring next to FIG. 4B, a form 410 is shown. The form 410 includes the frame 402 and the body 404. The body 404 here now includes a sub-form 412. The sub-form 412 includes fields for receiving input used to create a new user. The fields of the sub-form 412 include a first field 414 for receiving a first name of the new user, a second field 416 for receiving a last name of the new user, an email address field 418 for receiving an email address of the new user, a password field 420 for receiving a password of the new user, a phone number field 422 for receiving a phone number of the new user, a team field 424 for receiving an indication of a team for the new user, and a role field 426 for receiving an indication of a role for the new user. Certain ones of the fields within the sub-form 412 may be configured to receive text input from a user of the software application. Other ones of the fields within the sub-form 412 may be configured to display a list of options for the user of the software application to select from.

The form 400 represents a “create a team” page of the software application and is used to create a team. The form 410 represents a “create a user” page of the software application and is used to create a user. As used herein, a team refers to a group of users of the software application who share tasks and/or patient pools (e.g., as pulled from electronic medical records using an application programming interface (API)). As such, in a medical context, a team may therefore be defined to be or otherwise include one or more users selected to access a pool of common patients and tasks by the team administrator.

Team inclusion may be determined by request and approval by the team administrator with administrative security privileges (e.g., through email). Once a new user is created using the form 410, the new user (or another user of the software application) may use the form 400 to cause the new user to join a team, such as by the new user requesting access to the team. By requesting access to join a team, a user is requesting access to shared task lists and patients.

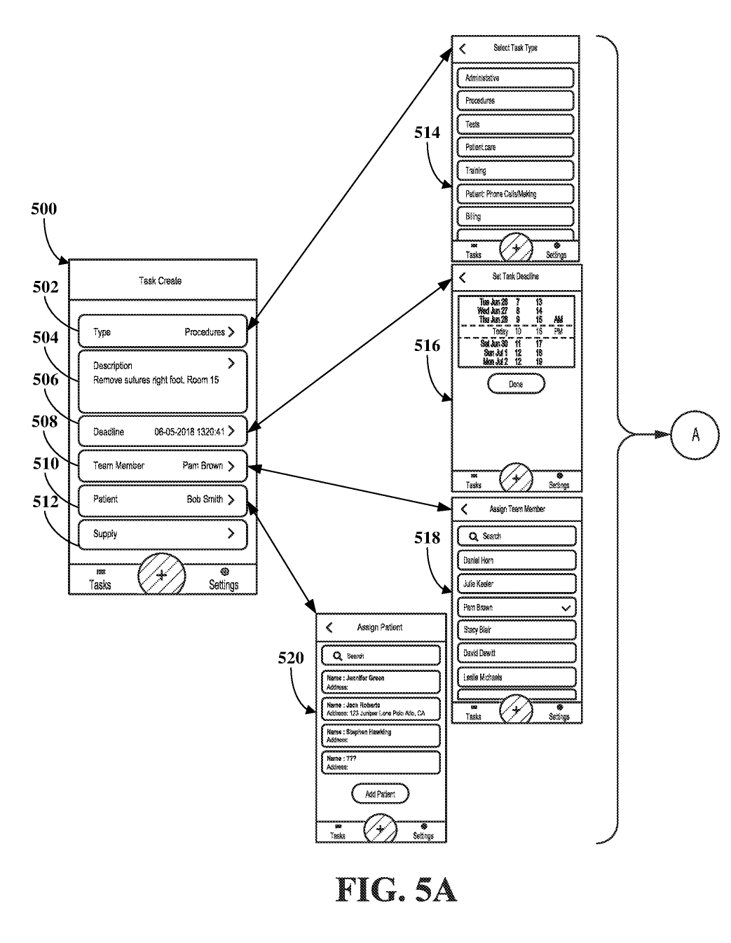

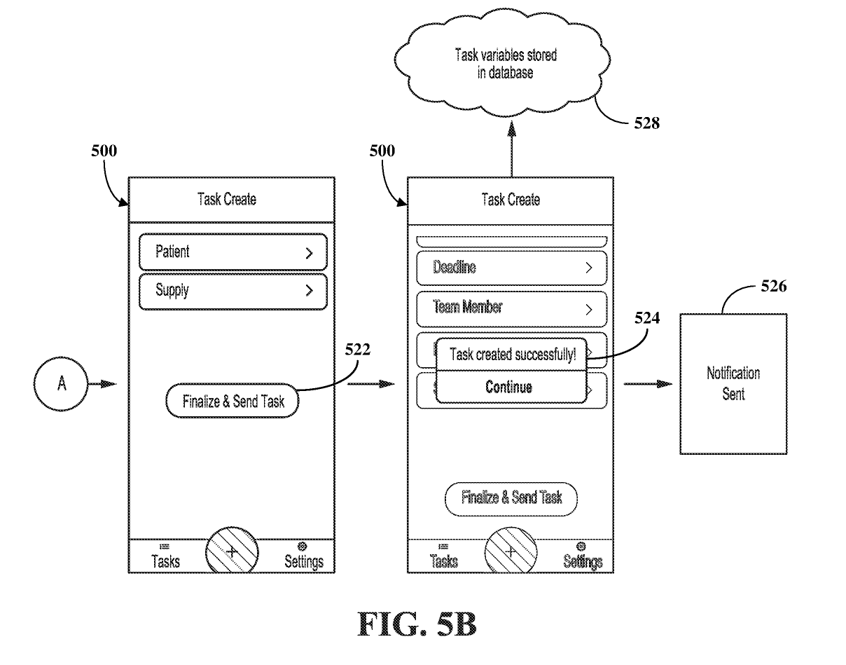

FIGS. 5A-B are illustrations of an example of a workflow for task creation using a software application for task management and communication, which may, for example, be the software application 104 shown in FIG. 1. Referring first to FIG. 5A, the workflow for task creation begins with user action with respect to a first GUI 500. The first GUI 500 represents a selection of or request for entry of information used to create a task. The first GUI 500 includes a number of elements that, when interacted with by a user, allow for the configuration of certain information for use in creating the task.

For example, the first GUI 500 includes a first element 502 for configuring a task type, a second element 504 for configuring a task description, a third element 506 for configuring a task deadline, a fourth element 508 for configuring an assigned team member, a fifth element 510 for configuring a named patient, and a sixth element 512 for configuring a supply. Some or all of the elements 502-512, when interacted with, advance the workflow by causing the software application to display a new GUI.

For example, selecting the first element 502 causes a second GUI 514 to be displayed. The second GUI 514 includes fields used to configure the task type. For example, the fields within the second GUI 514 can indicate a type of the task to be created, which may be administrative, procedural, test, patient care, training, patient phone calls, billing, or other types. In another example, selecting the third element 506 causes a third GUI 516 to be displayed. The third GUI 516 allows the user to select a date and time by which the task is to be completed.

In yet another example, selecting the fourth element 508 causes a fourth GUI 518 to be displayed. The fourth GUI 518 includes a list of team members (e.g., users who have been added to a team) who may be selected and assigned to the task. In yet another example, selecting the fifth element 510 causes a fifth GUI 520 to be displayed. The fifth GUI 520 includes a list of patients (or, in a non-medical context, customers or other persons to be assisted in connection with the performance of the task) who may be selected.

In some cases, the configurations made using some or all of the second GUI 514, the third GUI 516, the fourth GUI 518, or the fifth GUI 520 may be customized, such as where the presented options do not accurately reflect the goals for the new task.

Referring next to FIG. 5B, once the configurations are made using the elements 502-512 of the first GUI 500 (such as by accessing other GUIs, for example, the second GUI 514, the third GUI 516, the fourth GUI 518, and/or the fifth GUI 520), the user may select a finalize element 522 within the first GUI 500. Selecting the finalize element 522 causes the configurations made using the elements 502-512 to be used to create a new task.

Upon the creation of the new task, a pop-up element 524 is displayed on the first GUI 500 to reflect that the new task has been successfully created. As a result of the creation of the new task, a notification 526 is sent to one or more recipients to notify those one or more recipients as to the new task. The notification may, for example, be an email, an SMS message, or another written or electronic notification or message. Additionally, as a result of the creation of the new task, task variables 528 representing the configurations used to create the new task are stored in a database (e.g., the database 106 shown in FIG. 1), such as for later representing a record of the task within the software application or for otherwise later modifying the record of the task using the software application.

The workflow shown in FIGS. 5A and 5B may be used to create a new task. The creation of a new task using the workflow shown in FIGS. 5A and 5B enables users of the software application to delegate tasks to a team member or the user himself or herself, set a deadline date, set a deadline time, describe a task, and associate the task with a patient (e.g., in a medical context). The patient association entry, the deadline date, and the deadline time entries may, in some cases, be optional entries. A voice activation feature may be implemented to allow users to translate voice to text for some or all of the fields used to receive input on one or more of the first GUI 500, the second GUI 514, the third GUI 516, the fourth GUI 518, or the fifth GUI 520.

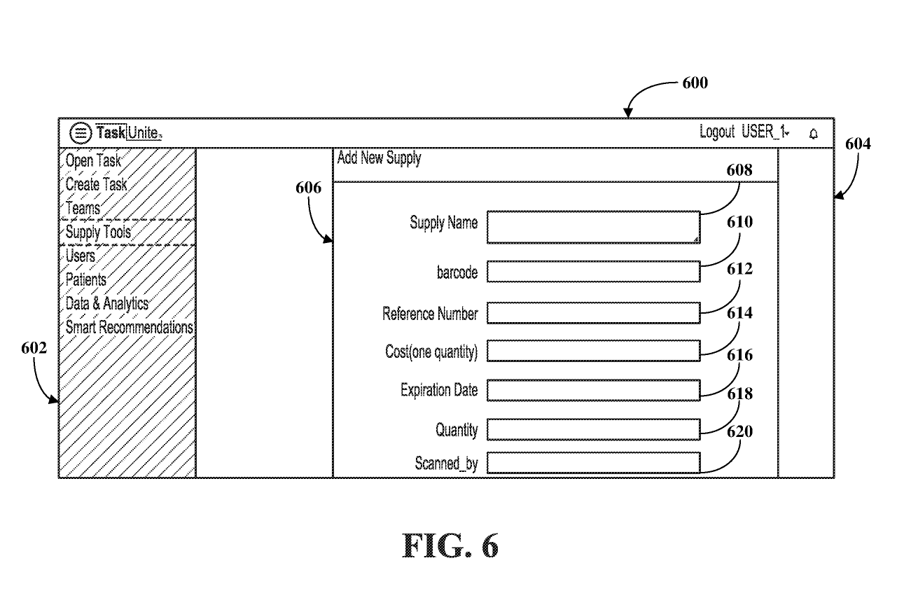

FIG. 6 is an illustration of an example of a form 600 for requesting supplies within a software application for task management and communication, which may, for example, be the software application 104 shown in FIG. 1. The form 600 includes a frame 602 and a body 604, which may, for example, be the frame 402 and the body 404 shown in FIGS. 4A and 4B. The body 604 includes a sub-form 606. The sub-form 606 includes field for receiving input used to create a new supply. The supply may be a product or other tangible or intangible item to use to perform a task. Creating a new supply can include requesting a new supply, indicating a use (or intended use) of a supply, or both.

For example, the sub-form 606 may include a first field 608 for a supply name, a second field 610 for a barcode or other code number of the supply, a third field 612 for a reference number for the supply, a fourth field 614 for a cost (e.g., at one quantity) of the supply, a fifth field 616 for an expiration date for the supply, a sixth field 618 for a quantity of the supply, and a seventh field 620 for an identifier or username of a user who scanned the supply.

The form 600 may be accessed after a corresponding task is created (e.g., based on the workflow described with respect to FIGS. 5A and 5B). After the task is created, the user to whom the task is assigned may access the form 600, such as responsive to authentication (e.g., using a personal identification number, a fingerprint or other biometric measure, etc.). The user may then use the form 600 to indicate the supplies used to perform the task (e.g., in the context of a medical treatment of a patient, intravenous tubing, gauze, needles, etc.). The user may enter the information about each such supply manually, such as by typing the information, or using some automated mechanism, for example, by scanning a barcode or other code which may be scanned and which is related to the supply.

Upon entering information within the fields 608-620, a supply record is created to indicate the information about the supply. The supply record may be stored in a database (e.g., the database 106 shown in FIG. 1), such as for later viewing or modification using the software application. An indication that the supply record has been created may also be sent to another user, such as a superior of the user who created the supply record using the form 600. For example, a notification that the supply record has been created may be sent to another user by email, SMS message, or another notification or message system.

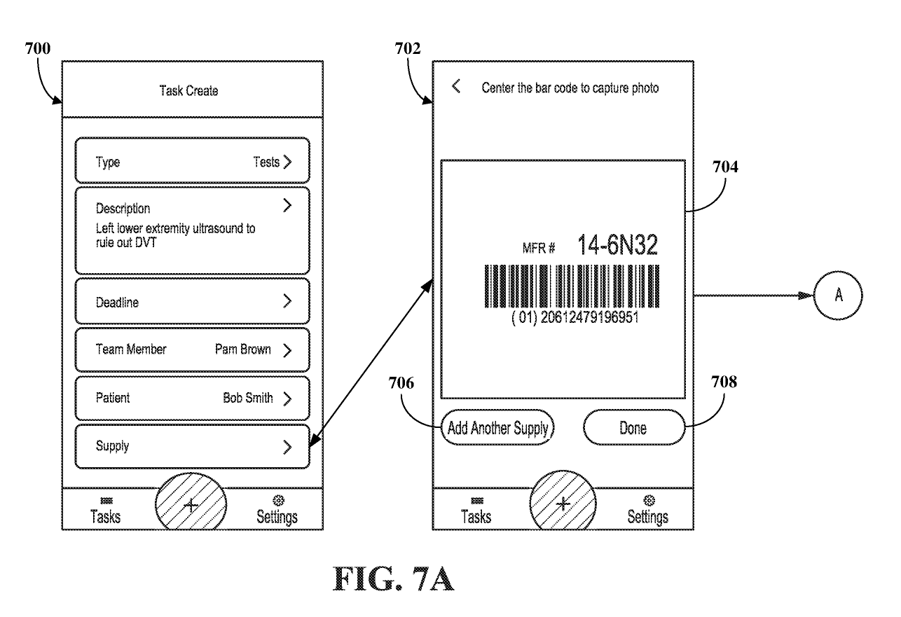

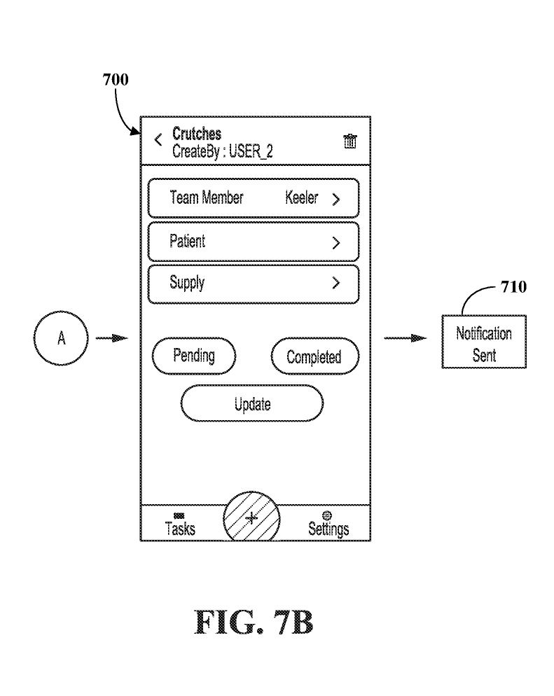

FIGS. 7A-B are illustrations of an example of a workflow for task response using a software application for task management and communication, which may, for example, be the software application 104 shown in FIG. 1. Referring first to FIG. 7A, the workflow for task response begins with user action with respect to a first GUI 700. The first GUI 700 represents a selection of or request for entry of information used to create a task. The first GUI 700 includes a number of elements that, when interacted with by a user, allow for the configuration of certain information for use in creating the task. For example, the first GUI 700 may be the first GUI 500 shown in FIGS. 5A and 5B.

Interacting with the element labeled as or described in connection with a supply or supplies causes a second GUI 702 to be displayed. The second GUI 702 includes elements used to input information for identifying supplies (e.g., which have been used to perform a task, which are requested so as to perform the task, or both). For example, the second GUI 702 includes a scanning element 704 which displays a real-time or near real-time video feed from an image sensor (e.g., of a camera) of a computing device on which the software application is running (e.g., a mobile device, such as a cell phone).

A user may focus the image sensor at a bar code or other code which can be scanned. That bar code or other code may then be displayed, such as in real-time or near real-time, within the scanning element 704. The software application may include functionality for processing data displayed within the scanning element 704, or for otherwise processing data which may be scanned or otherwise identified using an image sensor of a computing device. For example, the software application may include functionality for identifying a bar code or other code scanned using the image sensor and processing the identified bar code or other code to obtain data associated therewith. The data associated with an identified bar code or other code may then be used to update supply records, such as stored within a database accessible using the software application (e.g., the database 106 shown in FIG. 1).

The second GUI 702 includes additional elements, including an add element 706 and a completion element 708. The add element 706 may be interacted with by a user to cause the software application to allow another supply to be identified and processed in connection with the task, such as based on the functionality associated with the scanning element 704. The completion element 708 may be interacted with by the user to indicate that no further supplies are to be identified and processed in connection with the task.

Referring next to FIG. 7B, the first GUI 700 is shown with further elements, such as which may be displayed at a lower region of the first GUI 700. For example, the first GUI 700 may represent options for modifying the status and/or configurations of a task record. As such, a user may interact with one or more elements of the first GUI 700 to indicate whether the task remains pending, whether the task has been completed, or whether the task record associated with the task is to be updated.

A notification 710 is sent to one or more recipients to notify those one or more recipients as to changes in supplies associated with the task (e.g., as identified by using the second GUI 702) and/or changes in a status of the task (e.g., such as to change the status from pending to completed or to indicate an update to the configurations of the task record, such as based on the changes in supplies). The notification may, for example, be an email, an SMS message, or another written or electronic notification or message. Task variables representing the configurations and/or status associated with the task within a database (e.g., the database 106 shown in FIG. 1) may also be updated.

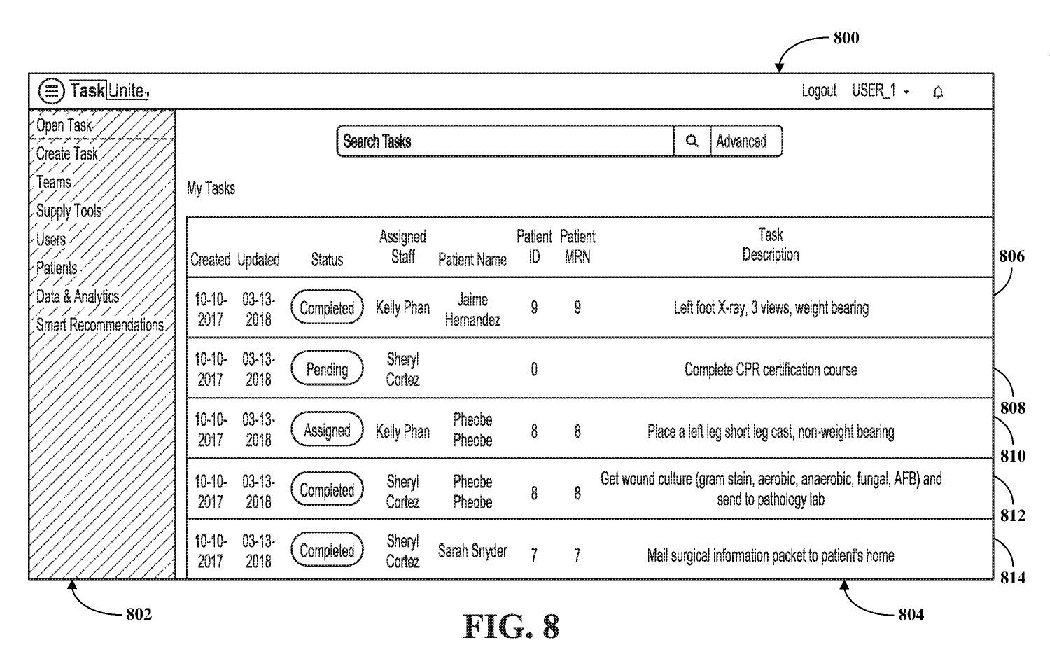

FIG. 8 is an illustration of an example of a form 800 for displaying task records within a software application for task management and communication, which may, for example, be the software application 104 shown in FIG. 1. The form 800 includes a frame 802 and a body 804. The frame 802 may, for example, be the frame 402 shown in FIGS. 4A and 4B. The body 804 includes a list of task records which have been created and processed using the software application.

For example, the record 806 refers to a task completed by user Kelly Phan, the record 808 refers to a pending task being performed by user Sheryl Cortez, the record 810 refers to an assigned (e.g., but not yet pending or otherwise in process) task to be performed by user Kelly Phan, the record 812 refers to a task completed by user Sheryl Cortez, and the record 814 refers to another task completed by user Sheryl Cortez.

The form 800 represents a task management page including task management user functionality within the software application. The sub-form 806 represents a table or other visualization of the tasks created and processed using the software application. For example, the information with the table or other visualization may indicate a date the task was created, a date the task was last updated, a status of the task, a user assigned to the task, a customer (e.g., patient) for whom the task is performed (e.g., by name, identifier, and/or other information), a description of the task, and/or other information or configurations associated with the task.

The interface with the sub-form can be used to generate statistical analysis comparing two or more of independent variables (e.g., customer name, user name, supply name, equipment name, task type, customer location, customer age, other customer information (e.g., patient medications, patient surgical history, etc.), or the like) versus dependent variables or covariates (e.g., time stamp metrics on task responses, frequency of supply use, frequency of supply reordering, or the like). Individual variables (e.g., customer name, date of birth, record number (e.g., medical record number), location (e.g., hospital or medical facility room number), other personal information (e.g., medications, diagnoses, surgical history, etc.), or the like) may be pulled from electronic records, such as by using an API.

The information stored within the table or other visualization represented by the sub-form 806 may also be sortable, such as by column, through the user interface. Furthermore, data stored in the table or other visualization represented by the sub-form 806 may also be searchable, such as for retrieval by keyword, customer demographics, user name, task name, supply name, date range, time range, or the like, or a combination thereof. The elements of the sub-form 806 or otherwise of the form 800 may enable a user to create a task, delegate a task, edit a task, associate a task with inventory and supplies, and/or provide analytics.

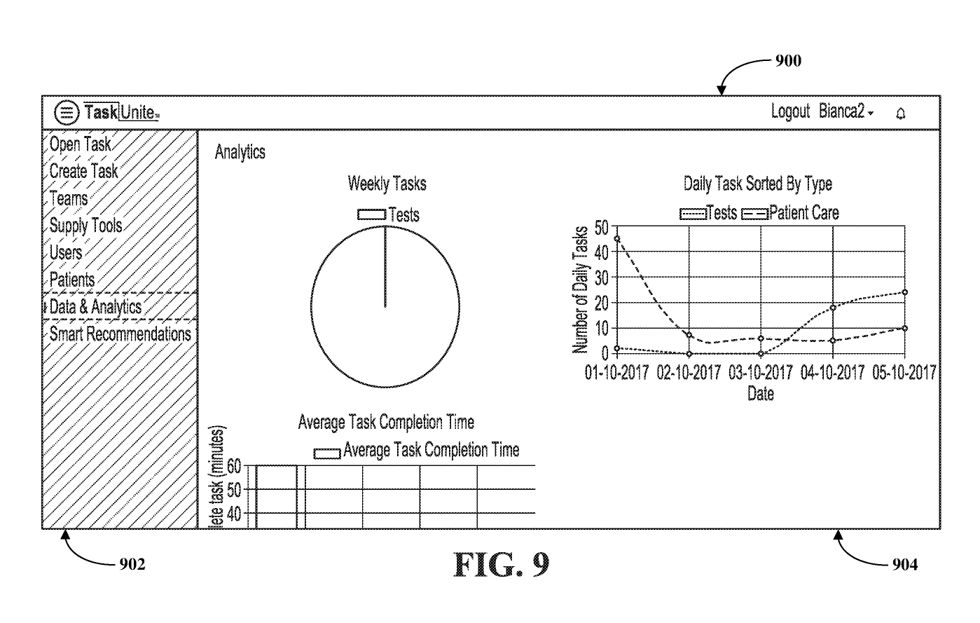

FIG. 9 is an illustration of an example of data and analytical functionality of a software application for task management and communication, which may, for example, be the software application 104 shown in FIG. 1. An aspect of the software application 104 includes a form 900 for displaying and/or interacting with analytical data from the software application. The form 900 includes a frame 902 and a body 604. The frame 902 may, for example, be the frame 402 shown in FIGS. 4A and 4B. The body 604 includes data and visual representations from the processing of task records and information relating to those task records.

The data and visual representations within the body 604 may represent analyses from the output of the tasks associated with those task records. For example, an analysis of tasks performed using the software application may be conducted to identify the percentage of those tasks which were completed by the respective deadline. The analyses shown within the body 604 may be conducted using or otherwise based on data stored within a database accessible using the software application (e.g., the database 106 shown in FIG. 1).

The form 900 may be available to certain users of the software application, such as based on the title of those users. For example, administrator account users may have the ability to conduct frequency analytics, parametric statistical tests, non-parametric statistical tests, and/or multivariate statistical tests, and then to further generate graphs, tables, and/or charts from the data. The data and visual representations within the body 904 of the form 900 can be exported, such as to a spreadsheet or other document which can be downloaded by the administrator.

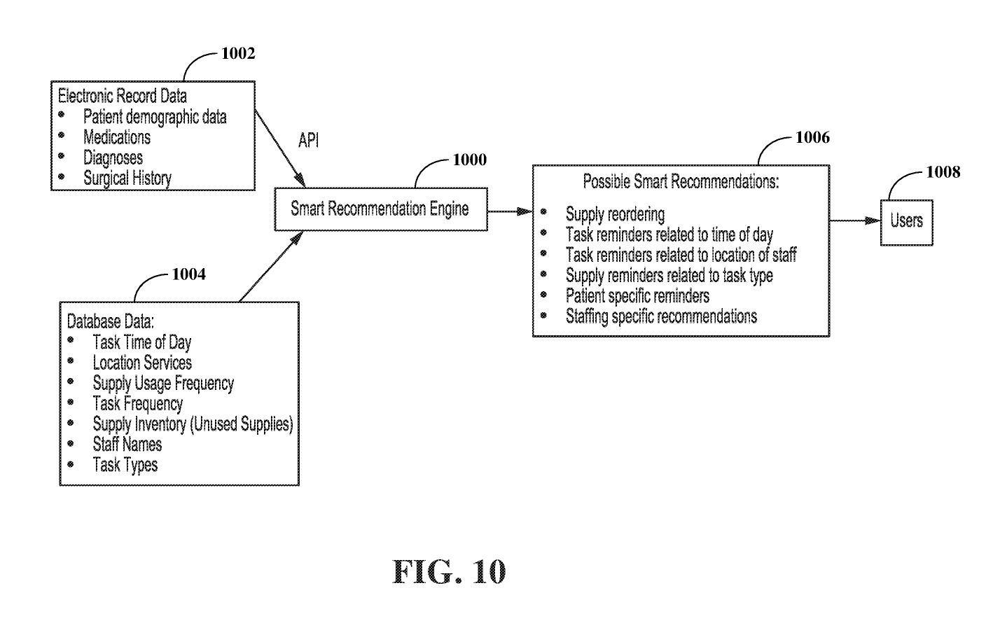

FIG. 10 is a diagram shown example functionality of a smart recommendation engine 1000 of a software application for task management and communication, which may, for example, be the software application 104 shown in FIG. 1. The smart recommendation engine 1000 represents machine learning or other cognitive or artificial intelligence functionality available to or otherwise of the software application.

In particular, the smart recommendation engine 1000 uses location services of users of the software application, electronic record data 1002 (e.g., electronic medical record data, such as data relating to patient demographics, diagnoses, medications, and surgical history), and database data 1004 accessible to the software application (e.g., task times, task types, time of day task completion, supply usage, and supply inventory) to generate task reminders without manual user intervention.

The task reminders may be notifications or other messages indicating tasks to perform. For example, possible smart recommendations output as task reminders by the smart recommendation engine 1000 include supply reordering tasks, task reminders related to a time of day, task reminders related to locations of users, supply reminders related to a task type, customer-specific reminders, and user-specific recommendations. The users 1008 of the software application receive the task reminders, such as within a form of the software application to which they have access for viewing task records.

The output of the smart recommendation engine 1006 may further be used to automate aspects of the software application. For example, the output of the smart recommendation engine 1006 may be used to re-order supplies when inventory is low, recommend supplies based on tasks frequently associated therewith, and recommend user task assignment based on optimizations identified using the machine learning or other cognitive or artificial intelligence aspects of the smart recommendation engine 1006.

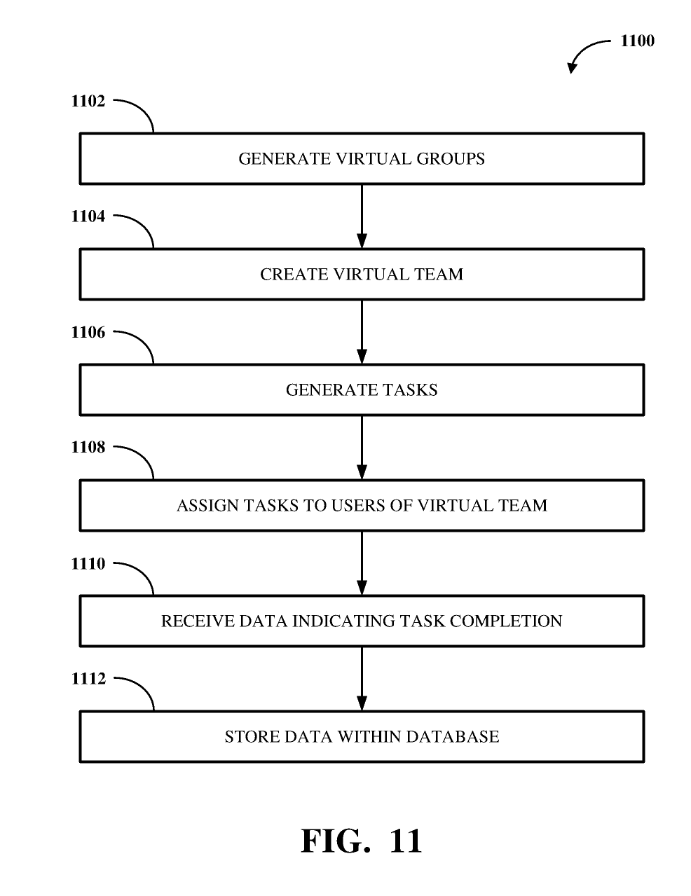

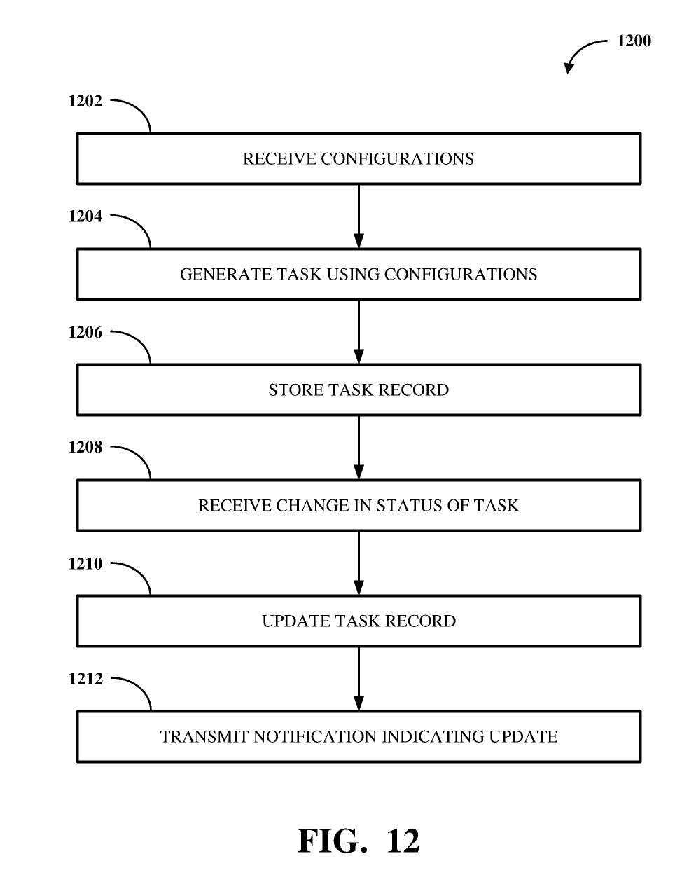

FIGS. 11 and 12 are flowcharts of examples of techniques 1100 and 1200, respectively, for task management and communication. The technique 1100 and/or the technique 1200, as well as other techniques as may be described herein, can be executed using computing devices, such as included within or otherwise using the systems, software, and devices described with respect to FIGS. 1-10. For example, the technique 1100 and/or the technique 1200 may be wholly or partially performed using the software application 104 shown in FIG. 1.

The technique 1100 and/or the technique 1200, as well as other techniques as may be described herein, can be performed, for example, by executing a machine-readable program or other computer-executable instructions, such as routines, instructions, or programs described according to Java, JavaScript, C++, or other such routines or instructions. The steps, or operations, of the technique 1100 and/or the technique 1200, as well as other techniques as may be described herein, or of a method, process, or algorithm described in connection with the implementations disclosed herein can be implemented directly in hardware, firmware, software executed by hardware, circuitry, or a combination thereof.

Although the technique 1100 and the technique 1200 as described herein are each shown as a series of operations for clarity, the steps, operations, or other functionality of the implementations of the technique 1100 and/or the technique 1200, or another technique, method, process, and/or algorithm described in connection with the implementations disclosed herein can be performed in various orders and/or concurrently. Additionally, operations in accordance with this disclosure can be performed with other operations not presented and described herein. Furthermore, one or more aspects of the systems and techniques described herein can be omitted.

Referring first to FIG. 11, at 1102, virtual groups are generated. The virtual groups are generated using health care system data stored within a database of a health care system. A virtual group refers to a defined grouping of patients of the health care system, such as based on locations of those patients within a hospital or other facility (e.g., floors or room numbers), doctors of those patients, common ailments or diagnoses, or the like. As such, each of the virtual groups includes at least one patient.

A record of each patient is stored within the database. The record may, for example, be an electronic medical record. The health care system may, for example, be a work location environment that includes users who perform patient health care or related services. The database of the health care system may be a database stored in a computing device located within that work location environment or a database accessed using a computing device located within that work location environment.

At 1104, a virtual team is created for each of the virtual groups. A virtual team refers to a team defined for assisting patients of a respective virtual group. For example, a virtual team may include doctors, nurses, staff, administrators, and/or other personnel who directly or indirectly service some or all of the patients of a virtual group. As such, each virtual team includes at least one user of the health care system. A virtual team may refer to each user of a given work location environment (e.g., a hospital or another medical facility). Alternatively, the virtual team may instead refer to some, but not all, users of the given work location environment (e.g., users working on one floor of a hospital or in one physical or other area of the other medical facility).

At 1106, tasks are generated. In particular, at least one task is generated for at least one patient within each of the virtual groups. A task may be generated using associated patient data (e.g., data stored within the database which is associated with the patient for whom the task is generated, such as electronic medical record data stored within the database). For example, a task may be generated based on configurations received as input from a user of the health care system. The configurations may refer to one or more of a task type, a task description, a task deadline, the user of the health care system, the patient of the health care system, or at least one supply to use to perform the task.

Responsive to generating the at least one task using the associated patient data stored within the database, a notification may be generated. The notification may indicate the generated at least one task or information associated therewith. For example, the notification can indicate an identifier of the task record generated in connection with the task, a name of the task, a deadline for completing the task, the patient for whom the task is to be performed, or more. The notification may then be transmitted to one or more users of the health care system.

At 1108, the tasks are assigned to users of the virtual teams corresponding to the virtual groups that includes the respective patients. Assigning a user to a task for a patient includes using a matching mechanism that processes, for the users within the virtual team corresponding to the virtual group that includes the patient, one or both of geofencing location coordinates of the users or availability status of those users. The geofencing location coordinates of the users may be determined based on a location of a work environment associated with the virtual team that includes the users

For example, the matching mechanism may reflect an automated process of a software application for selecting a user of the virtual team who is optimal for the task (e.g., based on a skill set of the user, a work experience or history of the user, etc.). The matching mechanism can use data associated with each user of the virtual team as input and process the data to select the user to whom the task is to be assigned. In some cases, however, the matching mechanism may be ignored or otherwise not used. For example, a user may be manually assigned to a task.

At 1110, subsequent to the assigning, data indicating a completion of a task by the user to which the task is assigned is received. The data may reflect data indicating that the task has been fully completed or partially completed. Thus, the data indicates some change in the status of the task. The data is received from a computing device of the user to whom the task is assigned. For example, the task may be generated using a first computing device, and the data indicating the completion of the task may be received from a second computing device.

At 1112, the data indicating the completion of the task by user is stored within the database. Storing the data within the database can include generating a new record within the database, wherein the new record indicates the data. Alternatively, storing the data within the database can include updating an existing record within the database according to the data.

In some implementations, the technique 1100 can include generating a task record for a task responsive to generating the task. For example, the task record may be stored as a new record within the database. The task record may, for example, indicate the user of the health care system to whom the task is assigned, the patient for whom the task is being performed, and/or other information about the task.

In some implementations, the technique 1100 can include transmitting a notification to a user of the health care system to indicate the completion of the task. For example, the completion of the task (e.g., the change in the status of the task) may be indicated to a user of the device used to generate the task (e.g., an administrator, manager, or other staff member of the virtual team who manages the tasks for the virtual team). The notification may be generated upon the identification of the change in status of the task (e.g., based on the data received which indicates the completion of the task).

In some implementations, the technique 1100 can include authenticating access to a user account of the health care system. For example, the technique 1100 can include receiving login information for authenticating a user and authenticating the user of the health care system by processing the login information against the data stored within the database.

In some implementation, the technique 1100 can include processing supplies used to perform a task assigned to a user of the health care system. For example, the technique 1100 can include identifying at least one supply to use to perform the at least one task by scanning a code associated with the at least one supply. Responsive to identifying the at least one supply, an inventory level of the at least one supply can be determined. Responsive to determining that the inventory level of the at least one supply is below a threshold value, an order for more of the at least one supply may be automatically produced, such as without manual user intervention. For example, producing the order can include using a pre-configured transaction arrangement (e.g., a script or the like) to request more of the respective supply or supplies. The threshold value may be the same or different for each supply. The threshold value may be manually configurable.

In some implementations, the technique 1100 can generate the at least one task and/or other tasks using a smart recommendation engine. For example, generating a task can include using a smart recommendation engine to generate the task based on associated patient data (e.g., electronic medical records) and other data (e.g., task-related data, such as configurations or the like) stored within the database. The smart recommendation engine processes the associated patient data and the other data stored within the database to produce a list of possible recommendations of the task.

The associated patient data used by the smart recommendation engine may, for example, include one or more of patient demographic data, medication data, diagnoses data, or surgical history data The other data stored within the database and used by the smart recommendation engine may, for example, include one or more of a task time, a location of services, a supply usage frequency, a task performance frequency, a supply inventory, list of users of the virtual groups, or a task type.

The possible recommendations may be reviewed and selected from by a user of the health care system. Alternatively, the smart recommendation engine can select one or more of the possible recommendations to output as a new task. For example, the smart recommendation engine can score the possible recommendations and select the one with the highest score, the ones having scores above a threshold value, or on other bases.

In some implementations, the technique 1100 can include generating analytical information. For example, records and data stored within the database of the health care system can be used to generate analytical information for one or both of a task or the user assigned to the task. The analytical information may, for example, indicate whether the task was timely completed, a percentage of tasks assigned to that user that have been timely completed, a total number of tasks in a related area (e.g., based on patient type or location) that have been generated and/or completed within a certain period of time, or more.

Referring next to FIG. 12, at 1202, configurations are received. The configurations may be received as first input received from a first client device. The configurations are used to generate a task. For example, the configurations may indicate one or more of a task type, a task description, a task deadline, the user of the health care system, the patient of the health care system, or at least one supply to use to perform the task to be generated. In some implementations, the indication of the user of the health care system may refer to the user of the health care system to which the task will be assigned. In some implementations, the configurations may be limited to indicating users of the health care system based on geofencing location coordinates associated with a work location environment of the users.

At 1204, a task is generated using the configurations. The task is indicative of one or more actions to perform with respect to a patient of the health care system. For example, the task may indicate that a user of the health care system has been selected to perform the one or more actions for the patient. At 1206, a task record associated with the task is stored. For example, generating the task using the configurations can include generating a task record representative of the task. In particular, the task record may indicate information about the user of the health care system to which the task is assigned. The task record is data that may be stored in a database or other data store. Storing the task record may thus include storing the task record in a database of the health care system.

At 1208, a change of status of the task is received. The change of the status of the task may be received as second input from a second client device. The change of status of the task may be data indicating that some or all of the task has been performed, such as by the user of the health care system to which the task was assigned. For example, the change of status of the task may indicate that the status of the task has changed from “pending” to “completed.” At 1210, the task record is updated according to the change of the status of the task. Updating the task record can include querying a database of the health care system for the task record and then modifying the found record based on the change of status.

At 1212, a notification indicating the update is transmitted. The notification represents that some change in status has occurred for the task, the particular reason for the change in status, or both. The notification may be transmitted to the first client device used to generate the task. Alternatively, the notification may be sent to another client device in communication with the health care system.

In some implementations, the technique 1200 may include using a smart recommendation engine to generate a second task based on the data stored within the database of the health care system. For example, the smart recommendation engine may process the data stored within the database to produce a list of possible recommendations of the second task. One of the tasks of the list of possible recommendations may then be selected as the second task, such as based on manual user selection, selection by machine learning, or both.

The implementations of this disclosure can be described in terms of functional block components and various processing operations. Such functional block components can be realized by any number of hardware or software components that perform the specified functions. For example, the described implementations can employ various integrated circuit components (e.g., memory elements, processing elements, logic elements, look-up tables, and the like), which can carry out a variety of functions under the control of one or more microprocessors or other control devices. Similarly, where the elements of the described implementations are implemented using software programming or software elements, the systems and techniques can be implemented with any programming or scripting language, such as C, C++, Java, JavaScript, assembler, or the like, with the various algorithms being implemented with a combination of data structures, objects, processes, routines, or other programming elements.

Functional aspects can be implemented in algorithms that execute on one or more processors. Furthermore, the implementations of the systems and techniques could employ any number of conventional techniques for electronics configuration, signal processing or control, data processing, and the like. The words “mechanism” and “element” are used broadly and are not limited to mechanical or physical implementations, but can include software routines in conjunction with processors, etc.

Likewise, the terms “mechanism,” “module,” or “monitor” as used herein and in the figures may be understood as corresponding to a functional unit implemented using software, hardware (e.g., an integrated circuit), or a combination of software and hardware. In certain contexts, such mechanisms, modules, or monitors may be understood to be a processor-implemented software mechanism, processor-implemented software module, or processor-implemented monitor that is part of or callable by an executable program, which may itself be wholly or partly composed of such linked mechanisms, modules, or monitors.

The terminology used in this disclosure is for the purpose of describing particular implementations and is not intended to be limiting. As used herein, the term “and/or” includes some or all possible combinations of one or more of the associated listed items. As used herein, the similar forms “a,” “an,” and “the” are intended to include the plural forms as well as the singular forms, unless the context clearly indicates otherwise. As used herein, the terms “comprises” and/or “comprising,” when used throughout this specification, specify the presence of stated features, steps, operations, elements, and/or components, but do not preclude the presence or addition of one or more other features, steps, operations, elements, components, and/or groups thereof.

Implementations or portions of implementations of the above disclosure can take the form of a computer program product accessible from, for example, a computer-usable or computer-readable medium. A computer-usable or computer-readable medium can be any device that can, for example, tangibly contain, store, communicate, or transport a program or data structure for use by or in connection with any processor. The medium can be, for example, an electronic, magnetic, optical, electromagnetic, or semiconductor device. Other suitable mediums are also available. Such computer-usable or computer-readable media can be referred to as non-transitory memory or media, and can include volatile memory or non-volatile memory that can change over time. A memory of an apparatus described herein, unless otherwise specified, does not have to be physically contained by the apparatus, but is one that can be accessed remotely by the apparatus, and does not have to be contiguous with other memory that might be physically contained by the apparatus.

While this disclosure has been described in connection with certain implementations, it is to be understood that this disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. An apparatus for health care task management and communication, the apparatus comprising:
a server device including a processor, a memory, a storage, and a network interface,
wherein the storage includes a database that stores data associated with application software for a health care system,
wherein the processor executes instructions stored in the memory to perform operations of the application software, the operations including:
receiving, using the network interface, first input from a first client device, the first input indicating configurations to use to generate a task;
generating the task using the configurations, wherein the task is indicative of one or more actions to perform with respect to a patient of the health care system;
responsive to generating the task, storing a task record associated with the task within the database, wherein the task record indicates a user of the health care system to which the task is assigned;
subsequent to storing the task record within the database, receiving second input from a second client device, the second input indicating a change in status of the task;
updating the task record according to the second input; and
transmitting, using the network interface, a notification indicative of the updated task record to the first client device.

2. The apparatus of claim 1, wherein the instructions include instructions to perform further operations of the application software including:
receiving login information for authenticating the user of the health care system; and
authenticating the user of the health care system by processing the login information against the data stored within the database.

3. The apparatus of claim 1, wherein the configurations indicate one or more of a task type, a task description, a task deadline, the user of the health care system, the patient of the health care system, or at least one supply to use to perform the task.

4. The apparatus of claim 3, wherein receiving the first input from the first client device includes receiving scanned data from the first client device, the scanned data associated with the at least one supply, the scanned data obtained using an image sensor of the first client device.

5. The apparatus of claim 3, wherein the indication of the user of the health care system refers to the user of the health care system to which the task is assigned, wherein the configurations are limited to indicating users of the health care system based on geofencing location coordinates associated with a work location environment of the users.

6. The apparatus of claim 1, wherein the task is a first task, wherein the instructions include instructions to perform further operations of the application software including:
using a smart recommendation engine to generate a second task based on the data stored within the database, wherein the smart recommendation engine processes the data stored within the database to produce a list of possible recommendations of the second task.

7. A method for health care task management and communication, the method comprising:
generating virtual groups using health care system data stored within a database of a health care system, wherein each of the virtual groups includes at least one patient, wherein a record of each patient is stored within the database;
creating a virtual team for each of the virtual groups, each virtual team including at least one user of the health care system;
for at least one patient within each of the virtual groups, generating at least one task using associated patient data stored within the database;
assigning the at least one task for the patient to a user of the virtual team corresponding to the virtual group that includes the patient, wherein the assigning uses a matching mechanism that processes, for the users within the virtual team corresponding to the virtual group that includes the patient, one or both of geofencing location coordinates of the users or availability status of the users;
subsequent to the assigning, receiving data indicating a completion of the at least one task by the user to which the at least one task is assigned; and
storing the data indicating the completion of the at least one task by the at least one user within the database.

8. The method of claim 7, further comprising:
responsive to generating the at least one task using the associated patient data stored within the database, generating a notification indicative of the generated at least one task; and
transmitting the notification to one or more users of the health care system.

9. The method of claim 8, wherein storing the data indicating the completion of the at least one task by the at least one user within the database comprises:
updating a task record associated with the at least one task according to the completion of the at least one task;
responsive to the updating, generating a new notification indicative of the completion of the at least one task; and
transmitting the notification to the one or more users of the health care system.

10. The method of claim 7, further comprising:
identifying at least one supply to use to perform the at least one task by scanning a code associated with the at least one supply; and
responsive to identifying the at least one supply, determining an inventory level of the at least one supply; and
responsive to determining that the inventory level of the at least one supply is below a threshold value, automatically producing an order for more of the at least one supply.

11. The method of claim 7, wherein generating the at least one task using the associated patient data stored within the database comprises:
using a smart recommendation engine to generate the at least one task based on the associated patient data and other data stored within the database, wherein the smart recommendation engine processes the associated patient data and the other data stored within the database to produce a list of possible recommendations of the at least one task.

12. The method of claim 11, wherein the associated patient data includes one or more of patient demographic data, medication data, diagnoses data, or surgical history data.

13. The method of claim 11, wherein the other data stored within the database includes one or more of a task time, a location of services, a supply usage frequency, a task performance frequency, a supply inventory, list of users of the virtual groups, or a task type.

14. The method of claim 7, further comprising:
using records and other data stored within the database to generate analytical information for one or both of the at least one task or the user assigned to the at least one task.

15. The method of claim 7, wherein the geofencing location coordinates of the users are determined based on a location of a work environment associated with the virtual team that includes the users.

16. A non-transitory computer readable medium comprising instructions that, when executed by a processor, cause the processor to perform operations for health care task management and communication, the operations comprising:
defining a virtual group including patients of a health care system;
generating a task associated with at least one patient of the virtual group;
assigning the task to a user of the health care system using a matching mechanism; and
receiving information indicating a completion of the task by the user.

17. The non-transitory computer readable medium of claim 16, wherein the operations for generating the task associated with the at least one patient of the virtual group comprise:
using, to generate the task, configurations indicating one or more of a task type, a task description, a task deadline, the user of the health care system, the patient of the health care system, or at least one supply.

18. The non-transitory computer readable medium of claim 16, wherein the task is a first task, the operations further comprising:
using a smart recommendation engine to generate a second task based on data stored within a database of the health care system, wherein the smart recommendation engine processes the data stored within the database to produce a list of possible recommendations of the second task.

19. The non-transitory computer readable medium of claim 16, the operations further comprising:
responsive to generating the task, generating a notification indicative of the task; and
transmitting the notification to one or more users of the health care system.

20. The non-transitory computer readable medium of claim 16, the operations further comprising:
identifying at least one supply to use to perform the at least one task by scanning a code associated with the at least one supply; and
responsive to identifying the at least one supply, determining an inventory level of the at least one supply; and
responsive to determining that the inventory level of the at least one supply is below a threshold value, automatically producing an order for more of the at least one supply.
